(19) United States
(12) Patent Application Publication
(10) Pub. No.: US 2016/0106055 A1
(43) Pub. Date: Apr. 21, 2016

(54) Ultra-Low Trypsin Inhibitor Soybean and Methods of Making Thereof

(71) Applicant: SCHILLINGER GENETICS, INC., West Des Moines, IA (US)

(72) Inventor: John A. Schillinger, Cumming, IA (US)

(73) Assignee: SCHILLINGER GENETICS, INC., West Des Moines, IA (US)

(21) Appl. No.: 14/920,865

(22) Filed: Oct. 22, 2015

Related U.S. Application Data
(63) Continuation-in-part of application No. 13/579,087, filed on Aug. 15, 2012.
(60) Provisional application No. 61/314,919, filed on Mar. 17, 2010.
(60) Provisional application No. 61/310,233, filed on Mar. 3, 2010.

(51) Int. Cl. A01H 5/10; C12N 15/82; A01H 1/02

(52) U.S. Cl. CPC A01H 5/10; A01H 1/02; C12N 15/8251

(57) ABSTRACT

Soybean seed, plants, and products therefrom having an ultra-low trypsin inhibitor phenotype with no additional chemical or physical heat treatment. Also disclosed are soybeans having the ultra-low trypsin inhibitor phenotype due to the presence of the Kunitz allele and three additional alleles. Also disclosed are soybean seed and plants having a unique content of trypsin inhibitor units and lineage, where said phenotype is due to the interaction or mutations of one or more genes. Also disclosed are plants and plant parts derived from growing the soybean seed herein.

### CROSS-REFERENCE TO RELATED APPLICATIONS

This application is a Continuation-in-Part of U.S. application Ser. No. 13/579,087 filed on Aug. 15, 2012 which is a U.S. National Phase application claiming priority to PCT Application PCT/US2011/027035 filed Mar. 3, 2011 which claims priority to both U.S. provisional Patent Application No. 61/310,233 filed Mar. 3, 2010, and U.S. provisional Patent Application No. 61/314,919 filed Mar. 17, 2010, all of which are incorporated herein by reference in their entirety.

### BACKGROUND

Soybean, Glycine max (L.) Merr., is an important and valuable field crop. Thus, a continuing goal of soybean plant breeders is to develop stable, high yielding soybean cultivars that are agronomically sound with beneficial traits.

The foregoing examples of the related art and limitations related therewith are intended to be illustrative and not exclusive. Other limitations of the related art will become apparent to those of skill in the art upon a reading of the specification.

### SUMMARY

The following embodiments and aspects thereof are described and illustrated in conjunction with products and methods, which are meant to be exemplary and illustrative, not limiting in scope. In various embodiments, one or more of the above-described problems have been reduced or eliminated, while other embodiments are directed to other improvements.

One embodiment discloses an untreated and unheated soybean seed that expresses a diminished amount of an endogenous 2S-Protein, wherein the endogenous 2S-Protein is a protease trypsin inhibitor, and wherein the trypsin inhibitor units within said seed is between 4,800 trypsin inhibitor units and 15,122 trypsin inhibitor units.

Another embodiment discloses a soybean seed, wherein the total soluble 2S-Protein is approximately 6.85% or less, and wherein the amount of Kunitz trypsin inhibitor protein is approximately 3.01% or less of the total soluble 2S-Protein.

Another embodiment discloses a soybean seed, wherein the amount of Bowman-Birk inhibitor protein is approximately 1.31% or less of the total soluble 2S-Protein.

Another embodiment discloses methods of producing a commodity plant product, comprising obtaining a plant, or a part thereof, wherein the trypsin inhibitor units within said seed of said plant is between 4,800 trypsin inhibitor units and 15,122 trypsin inhibitor units, wherein the commodity plant product is protein concentrate, protein isolate, soybean hulls, meal, flour or oil and producing said commodity plant product therefrom.

Another embodiment discloses a method for introducing the combination of the Kunitz allele and three alleles associated with conferring a ultra-low trypsin inhibitor phenotype to a soybean plant lacking said alleles comprising: obtaining a first soybean plant wherein said soybean plant contains a genome comprising the Kunitz allele and three alleles associated with conferring a ultra-low trypsin inhibitor phenotype, wherein a representative sample of said alleles is present in ATCC accession number is PTA-10684; crossing said first soybean plant with a second soybean plant, wherein said second soybean plant lacks said alleles; selecting for progeny plants that have low trypsin inhibitor unit content; and backcrossing said progeny plants to said first parent plant until the progeny plants can be identified as exhibiting the Kunitz allele and three alleles associated with the ultra-low trypsin inhibitor phenotype.

Another embodiment discloses an untreated and unheated soybean seed that expresses a diminished amount of a protease trypsin inhibitor, wherein the amount of trypsin inhibitor units within said seed is between 4,800 trypsin inhibitor units and 15,122 trypsin inhibitor units, and wherein said diminished amount of a protease trypsin inhibitor is due to the presence of the Kunitz allele and at least three additional alleles, wherein a representative sample of said alleles is present in ATCC accession number is PTA-10684.

Another embodiment discloses an untreated and unheated soybean seed that expresses a diminished amount of a protease trypsin inhibitor, wherein the amount of trypsin inhibitor units within said seed is between 4,800 trypsin inhibitor units and 15,122 trypsin inhibitor units, and wherein said diminished amount of a protease trypsin inhibitor is due to the presence of the Kunitz allele and three additional alleles. A diminished amount of means that the amount of trypsin inhibitor is significantly reduced in a soybean seed when compared with a soybean seed not containing the Kunitz allele and the three additional alleles of the present invention that reduce the amount of trypsin inhibitor in a soybean seed.

Another embodiment discloses the ultra-low trypsin inhibitor phenotype, wherein said phenotype is comprised of the Kunitz allele and three additional alleles, wherein said alleles are comprised of a dominant allele and two additive alleles.

Other aspects, features and advantages will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

As used herein, “sometime” means at some indefinite or indeterminate point of time. So for example, as used herein, “sometime after” means following, whether immediately following or at some indefinite or indeterminate point of time following the prior act.

Various embodiments are set forth in the Detailed Description as provided herein and as embodied by the claims. It should be understood, however, that this Summary does not contain all of the aspects and embodiments, is not meant to be limiting or restrictive in any manner, and that embodiment(s) as disclosed herein is/are understood by those of ordinary skill in the art to encompass obvious improvements and modifications thereto.

In addition to the exemplary aspects and embodiments described above, further aspects and embodiments will become apparent by study of the following descriptions.

### BRIEF DESCRIPTION OF THE DRAWINGS

FIG. 1 shows a breakdown the 2S, 7S, and 11S proteins and shows, for the present disclosure, that the BBI, KTI, and other 2S-Proteins are collectively termed the total soluble protein (TSP), which is then used as the sample to determine the amount of trypsin inhibitor units (TIU).

### DEFINITIONS

In the description and tables herein, a number of terms are used. In order to provide a clear and consistent understanding of the specification and claims, including the scope to be given such terms, the following definitions are provided:

Allele. An allele is any of one or more alternative forms of a gene which relate to one trait or characteristic. In a diploid cell or organism, the two alleles of a given gene occupy corresponding loci on a pair of homologous chromosomes.

Backcrossing. Backcrossing is a process in which a breeder repeatedly crosses hybrid progeny back to one of the parents, for example, a first generation hybrid F₁ with one of the parental genotypes of the F₁ hybrid.

Cell. Cell as used herein includes a plant cell, whether isolated, in tissue culture or incorporated in a plant or plant part.

Coding Sequence. Refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

Cotyledon. A cotyledon is a type of seed leaf. The cotyledon contains the food storage tissues of the seed.

Derived From. The term “derived from” includes genes, nucleic acids, and proteins when they include fragments or elements assembled in such a way that they produce a functional unit. The fragments or elements can be assembled from multiple organisms provided that they retain evolutionarily conserved function. Elements or domains could be assembled from various organisms and/or synthesized partially or entirely, provided that they retain evolutionarily conserved function, elements or domains. In some cases the derivation could include changes so that the codons are optimized for expression in a particular organism.

Embryo. The embryo is the small plant contained within a mature seed.

Endogenous Protein. Refers to native protein normally found in its natural location in the plant.

Expression. The term “expression”, as used herein, refers to the transcription and stable accumulation of sense (mRNA) or antisense RNA derived from the nucleic acid fragment of the invention. Expression may also refer to translation of mRNA into a polypeptide.

Gene. As used herein, “gene” refers to a segment of nucleic acid. A gene can be introduced into a genome of a species, whether from a different species or from the same species, using transformation or various breeding methods.

Gene Silencing. The interruption or suppression of the expression of a gene at the level of transcription or translation.

Genotype. Refers to the genetic constitution of a cell or organism.

Kunitz allele. An allele of the Kunitz trypsin inhibitor gene, KTi3, containing nucleotides that differ from the wild-type gene at positions +481, +486, and +487, and result in a frameshift mutation causing the Kunitz phenotype as described in Orf and Hymowitz, J. Am. Oil Chern. Soc., 56:722-726 (1979) and Jofuku, et al., The Plant Cell, 1:427-435 (1989). The soybean variety, carrying only this Kunitz allele for reduced trypsin inhibitor activity, is referred to as the “Kunitz line.” These lines are readily available to the public.

Kunitz Phenotype. The trypsin inhibitor activity (in trypsin inhibitor units, TIU) found in soybeans carrying, as the only trypsin inhibitor gene mutation, the Kunitz allele, characterized as having at least a 30% reduction in trypsin inhibitor activity compared to commercial soybean lines having no mutations in trypsin inhibitor genes.

Linkage. Refers to a phenomenon wherein alleles on the same chromosome tend to segregate together more often than expected by chance if their transmission was independent.

Linkage Disequilibrium. Refers to a phenomenon wherein alleles tend to remain together in linkage groups when segregating from parents to offspring, with a greater frequency than expected from their individual frequencies.

Locus. A locus confers one or more traits such as, for example, male sterility, herbicide tolerance, insect resistance, disease resistance, waxy starch, modified fatty acid metabolism, modified phytic acid metabolism, modified carbohydrate metabolism and modified protein metabolism. The trait may be, for example, conferred by a naturally occurring gene introduced into the genome of the plant by backcrossing, a natural or induced mutation, or a transgene introduced through genetic transformation techniques. A locus may comprise one or more alleles integrated at a single chromosomal location.

Maturity Group. This refers to an agreed-on industry division of groups of varieties based on zones in which they are adapted, primarily according to day length or latitude. There are typically 13 maturity group categories. They consist of very long day length varieties (Groups 000, 00, 0), and extend to very short day length varieties (Groups VII, VIII, IX, X).

Non-transgenic mutation. A mutation that is naturally occurring or induced by conventional methods (e.g., exposure of plants to radiation or mutagenic compounds), not including mutations made using recombinant DNA techniques.

Phenotype. The detectable characteristics of a cell or organism.

Plant. As used herein, the term “plant” includes reference to an immature or mature whole plant, including a plant from which seed or grain or anthers have been removed. Seed or embryo that will produce the plant is also considered to be the plant.

Plant Parts. As used herein, the term “plant parts” (or a soybean plant, or a part thereof) includes but is not limited to protoplasts, leaves, stems, roots, root tips, anthers, pistils, seed, grain, embryo, pollen, ovules, cotyledon, hypocotyl, pod, flower, shoot, tissue, petiole, cells, meristematic cells, and the like.

Progeny. As used herein, includes an F₁ soybean plant produced from the cross of two soybean plants where at least one plant includes a soybean plant of the present invention and progeny further includes but is not limited to subsequent F₂, F₃, F₄, F₅, F₆, F₇, F₈, F₉, and F₁₀ generational crosses with the recurrent parental line.

Protein or Polypeptide. A “protein” or “polypeptide” is a chain of amino acids arranged in a specific order determined by the coding sequence in a polynucleotide encoding the polypeptide. Each protein or polypeptide has a unique function.

Quantitative Trait Loci (QTL). Quantitative trait loci (QTL) refer to genetic loci that control to some degree numerically representable traits that are usually continuously distributed.

Recombinant polynucleotide. The term recombinant polynucleotide refers to a polynucleotide which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

Regeneration. Regeneration refers to the development of a plant from tissue culture.

Single Gene Converted (Conversion). Single gene converted (conversion) plants refers to plants which are developed by a plant breeding technique called backcrossing wherein essentially all of the desired morphological and physiological characteristics of a variety are recovered in addition to the single gene transferred into the variety via the backcrossing technique or via genetic engineering.

Total Soluble Protein. Total soluble protein means the total amount of soluble protein located within, for example, the 2S storage protein.

Trypsin. Trypsin is a digestive enzyme, specifically, a pancreatic serine protease enzyme with substrate specificity based upon positively charged lysine and arginine side chains and is excreted by the pancreas. Trypsin aids in the digestion of food proteins and other biological processes.

Trypsin inhibitor units. Trypsin inhibitor units or abbreviated as TIU, is an assay measuring the quantity of trypsin inhibitor in a soybean seed or soybean product thereof. Measurement of trypsin inhibitor units is a technique well-known in the art.

Un-Heated. Means a soybean seed or plant that has not been physically treated with heat.

Un-Treated. Means a soybean seed or plant that has not been chemically treated.

Protease Inhibitors. protease inhibitors are molecules that inhibit the function of proteases.

Ultra-Low Trypsin Inhibitor Phenotype. The ultra-low trypsin inhibitor phenotype means a soybean seed having a phenotype of between 4, 800 trypsin inhibitor units and 15,122 trypsin inhibitor units.

### DETAILED DESCRIPTION OF THE EMBODIMENTS

The embodiments recited herein relate generally to the field of plant breeding and molecular biology and to soybean seeds and plant with an ultra-low content of trypsin inhibitor as measured by trypsin inhibitor units, TIU, and materials and methods for making such plants.

### Protein Content of Soybeans

Soybean protein is one of the highest quality of plant sources of protein. Soybean is made from soybean meal that have been dehulled and defatted. The amount of protein present in soybean seed is important because if the amount of protein can be increased, then the nutritional quality of the soybean itself, as well as other soybean products derived therefrom, can be increased.

### Plant Storage Proteins

Plant storage proteins are important for human nutrition, as about 70% of our protein demand is met directly or indirectly by the consumption of seeds. The storage proteins of legumes, including soybeans, are packaged into protein bodies which are formed by budding from the Endoplasmic Reticulum, and deposited in the cotyledon of the seed. In soybean, the storage proteins equate to between 40% and 50% of dry weight. Some protein bodies also contain proteins that act as a defense mechanism and protect the seeds from being eaten. For example, the seeds of some legumes, including soybeans, contain lectins, which bind to sugar residues in the intestine and interfere with the absorption of food. Some contain protease inhibitors that block the digestion of proteins by inhibiting proteinases in the animal digestive tract. Because of this, these protease inhibitors must be denatured—often times through cooking—before the legume is suitable for consumption.

The 2S-Proteins are a heterogeneous group of storage proteins encoded by multi-gene families. Their name is derived from their sedimentation coefficient of about 2 svedberg (S). The 2S-Proteins are widely distributed and packaged into protein bodies. Napin, the predominant storage protein in rapeseed, is an example of a 2S-Protein. The 2S-Proteins have related structures, and consist of large and small polypeptides linked by disulfide bonds. Additional storage proteins are also classified by their sedimentation coefficient, for example, there are 7S-Proteins and 11S-Proteins. While the 7S and 11S-Proteins are predominately globulins and impact nutrition, taste, and texture, the 2S-Proteins are both globulins and albumins. The 2S albumin (water soluble) storage proteins in soybeans are becoming increasingly interesting in that current literature indicates that the 2S storage protein is the location of where trypsin inhibitors can be found. Soybean seed proteins are an example of storage proteins that are widely used in human foods and animal feed and must normally be processed, via chemicals or heat, to remove or deactivate the protease inhibitors.

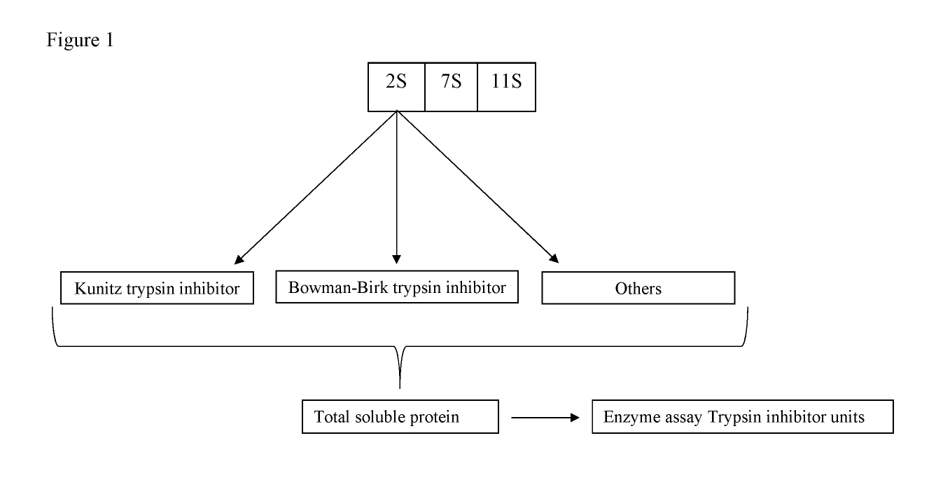

The albumin 2S-Proteins are further grouped into a prolamin superfamily that also includes the protease inhibitors, Kunitz trypsin inhibitor (KTI) and Bowman-Birk trypsin inhibitor (BBI). Both KTI and BBI are considered anti-nutrients because of their ability to inhibit digestive proteases in humans. Thus, for human consumption soybeans must undergo additional processing, often times by heat, to inactivate these inhibitors. Therefore, soybean varieties which are naturally low in Kunitz and Bowman-Birk inhibitors are desirable, since less processing is required. FIG. 1 is a diagram of the 2S-, 7S-, and 11S-Proteins and shows, for the present disclosure, that the BBI, KTI, and other 2S-Proteins are collectively termed the total soluble protein (TSP), which is then used as the sample to determine the amount of trypsin inhibitor units (TIU).

### Trypsin

Trypsin is an important digestive enzyme, particularly in certain species where ancillary enzymes, such as pepsin and chymotrypsin are present in relatively small amounts, or are absent. From an economic standpoint, the most important of these species are chickens, pigs, and calves (when the calves are sufficiently young that they have not developed a fully mature digestive system). In such animals, in particular, if the enzyme trypsin is in some way impaired in its functioning, there are a number of deleterious results. First, any food which is ingested by the animal is lowered in nutritive value because of a directly impaired capacity to digest it. Second, even in animals which contain other digestive enzymes in addition to trypsin, trypsin normally activates some of these enzymes and allows their participation in the process. A deficiency in trypsin thus results in a concomitant deficiency in these enzymes. Finally, in response to a perceived lack of adequate trypsin, the pancreas is induced to release more trypsin than it is easily capable of releasing, resulting in an “overwork” condition called pancreatic hypertrophy, which at best, results in morbidity and at worst, in death.

Kunitz trypsin inhibitor is an anti-nutritional and allergenic factor in soybeans that interferes with digestion and absorption of proteins when present in a diet. Genetic and biochemical studies of Kunitz trypsin inhibitor production in soybean lines have been carried out (please see for example de Moraes, R. M. A., et al., “Assisted selection by specific DNA markers for genetic elimination of the kunitz trypsin inhibitor and lectin soybean seeds. Euphytica, 149:221-226 (2006) and Natarajan, S., et al., J. of Plant Physiol., 164(6): 756-763 (2007)), and three related genes have been identified, with KTI3 encoding the predominant Kunitz trypsin inhibitor protein in cultivated soybean genotypes (Natarajan et al., 2006). Some specific DNA markers associated with loss of Kunitz production in certain soybean lines have been reported (de Moraes, R. M. A., et al., 2006).

The Kunitz phenotype refers to a specific trypsin inhibitor and is responsible for a reduction in total trypsin inhibition (measured in TIU, trypsin inhibitor units) by roughly a third the level of commercially available soybeans. The unique phenotype of the instant application is an additional, stepwise, reduction in total trypsin inhibitor. It is likely that this reduction is the response to a mutation or mutations in other trypsin inhibitors, such as the Bowman-Burk trypsin inhibitors.

The Bowman-Birk trypsin inhibitors represent a group of soybean trypsin inhibitors that are genetically distinct from the Kunitz trypsin inhibitors. There are thought to be 6 to 10 different genes belonging to the Bowman-Birk class of inhibitors in soybeans, some mutants of which have been investigated (e.g., Livingstone, et al., Plant Mol. Biol., 64:397-408 (2007). The Bowman-Birk inhibitors appear to make up most of the remaining 65-70% of trypsin inhibitor activity not accounted for by the Kunitz trypsin inhibitors.

Trypsin inhibition is an insurmountable problem when the ingested foodstuff contains large quantities of soybean materials which have not been subjected to proper treatment to destroy a soybean trypsin inhibitor which is capable of binding the endogenous trypsin in the animal ingesting the foodstuff, and in preventing it from carrying out its normal function. Hence, animal foods which are largely soybean based are currently treated by “cooking” to inactivate this protein. In conventional soy processing, the soybeans are dehulled using a wet process, wherein the water content, however, is purposely limited in order to reduce waste weight and in order to prevent interference with subsequent processing steps. The hulled soybeans are then extracted with hexane to remove the soybean oil for commercial use. After the hexane extraction, the soybean mulch is heated to inactivate the soybean trypsin inhibitor.

This inactivation process is conducted at considerable expense, and with imperfect results. The heating produces a decline in soybean trypsin inhibitor content. Therefore, after a time period which is optimum for the particular preparation in question, further heating becomes uneconomical and counterproductive, even though additional amounts of soybean trypsin inhibitor would be thereby inactivated. The resulting processed soybean meal is then used in animal feeds in a variety of forms, and is reduced in soybean trypsin inhibitor but still contains residual amounts. Please see U.S. Patent Publication No. 2012031675 for additional information on trypsin.

Protease inhibitors, such as the Kunitz trypsin inhibitor and the Bowman-Birk trypsin inhibitor are anti-nutritional proteins found in soybean seed, and various methods and techniques are commonly taken to reduce the amount of these protease inhibitors in seed. However, these methods and techniques frequently rely on the use of chemicals or physical heat to treat soybean and soybean products therefrom. The use of chemicals or heat not only denatures and inactivates these inhibitors in the seed, but also damages or destroys the protein content itself. The trypsin inhibitor content of soybean seed disclosed in the present application is due to the presence of one or more genes. Said genes can be tested using any suitable means, such as, for example, by obtaining a sample of the soybean plant or seed, and assaying the material for the presence of the mutant sequence by the use of general molecular techniques such as PCR, DNA hybridization using a nucleic acid probe, RFLP analysis, or nucleic acid sequencing methods.

Similarly, the presence of the Kunitz allele can be tested, for example, by obtaining a sample of the soybean plant or seed, and assaying the material for the presence of the mutant sequence by the use of general molecular techniques such as PCR, hybridization with a labeled nucleic acid probe, or nucleic acid sequencing methods.

In some embodiments of the current invention, non-transgenic mutations conferring a reduced trypsin inhibitor phenotype may comprise mutations in Kunitz alleles. In certain embodiments, the mutant Kunitz alleles are detected using genetic markers comprising polymorphisms within the Kunitz allele. In further aspects of the invention, plants with a reduced trypsin inhibitor phenotype may comprise one or more mutant alleles.

In some embodiments, non-transgenic mutations conferring a reduced trypsin inhibitor phenotype may comprise mutations in alleles of genes other than Kunitz genes, such as genes for the Bowman-Birk trypsin inhibitors.

In other embodiments, mutations conferring a Kunitz phenotype may comprise mutations in a gene encoding the Kunitz allele. In another embodiment, mutations conferring a Kunitz phenotype comprise mutations in the KTI3 gene (Kim et al., Theor. Appl. Genet., 121(4):751-60 (2010); Genbank Accession No. S45092).

### EXAMPLES

### Discovery, Development of the Ultra-Low Trypsin Inhibitor Phenotype

The ultra-low trypsin inhibitor phenotype was developed in 2006 by crossing soybean variety 435.TCS, known from molecular testing to have a wild-type copy of the Kunitz allele, and the Kunitz soybean line. Following this cross, lines were advanced to F₃ using the modified pod pick method. Single F₅ and F₆ plants were selected based on basic agronomics (plant type, stature, and structure). Selected F₇ lines were analyzed for total trypsin inhibitor content and the novel phenotype was discovered. The ultra-low trypsin inhibitor phenotype was not found in segregants from other genetic crosses, and the observed reduction in trypsin inhibitor activity is due to one or more mutations at one or more loci other than the Kunitz allele.

### Laboratory Protocol for TIU Determination

The laboratory protocols for determination of the amount of TIU in a sample is well-known in the art. Please see Stauffer, Clyde E. (1990) “Measuring TI Inhibitor in Soy Meal: Suggested Improvement in the Standard Method” Cereal Chem. 67(3): 296-302; Page et al., (2000) “Trypsin Inhibitor Activity Measurement: Simplifications of the Standard Procedure Used for Pea Seed: Crop Science. 40(5): 1482-1485; Sadasivam, et al., (1996) “In: Biochemical Methods for Agricultural Science” Wiley Eastern Ltd., New Delhi, India, pp: 187-188; and Kakaaade, et al., (1974) “Determination of Trypsin Inhibitor Activity of Soy Products: A Collaborative Analysis of An Improved Procedure” Cereal. 51: 376-382. The above techniques are illustrative and not limiting. For example, samples can exposed to trypsin to begin the cleaving process and incubated at 37° C. for about 15 minutes before undergoing spectral (spectrophotometer) analysis. Once an initial spectral analysis is complete, samples are left to sit for about 45 minutes at a temperature of 37° C. and then analyzed once again for amount of color change with correlates directly to TIU concentration present in the sample. Heat can be used in this step is to simulate the digestive conditions that the samples would encounter during typical digestion in the mammalian stomach where similar chemical digestion occurs. All other steps in the assay can be performed at room temperature which is about 23° C. Additionally, various solvents may be used in the TIU determination protocol, such as DMSO (Dimethyl Sulfoxide), which is similar to diethyl ether in that it is an aprotic solvent, but distinctly different because DMSO is polar while diethyl ether is considered nonpolar. Additional reagents listed above that can be used in the trypsin assay are HCl, NaOH, Tris-HCl, CaCl2, BAPNA, and optionally 30% acetic acid could be used to store the samples.

### Analysis of Lines for Ultra-Low Trypsin Inhibitor Phenotype

Table one below shows the trypsin inhibitor unit content, TIU, of various soybean lines analyzed in 2010. Trypsin inhibitor values were analyzed for 435.TCS, Kunitz, several commercial varieties, and novel lines designated 029K417, 029K418, 037K421, and 031K420, which were produced by crossing variety 435.TCS with the Kunitz soybean line (Table 1). The Kunitz and 435.TCS lines were grown in Illinois, the commercial varieties were grown in southern Illinois, and the new hybrid lines were grown in Iowa. As shown in Table 1, column 2, hybrid sub-lines derived from 435.TCS x Kunitz progeny exhibited a reduction in trypsin inhibitor values (column 3). These results demonstrate the unexpected ultra-low trypsin inhibitor phenotype characteristic of the Kunitz crossed with Schillinger proprietary soybean lines.

TABLE 1

| Line | Variety | TIU |
|---|---|---|
| Parent line | 435.TCS | 38000 |
| Parent line | Kunitz | 29000 |
| Commercial lines | 348.TCS | 49000 |
| | P93B82 | 50300 |
| | XC4510 | 46900 |
| Novel sub-lines of | 029K417-1 | 7100 |
| 029K417 | 029K417-2 | 6000 |
| | 029K417-3 | 6200 |
| | 029K417-4 | 8600 |
| | 029K417-5 | 4900 |
| | 029K417-6 | 5900 |
| | 029K417-7 | 5900 |
| | 029K417-8 | 5400 |
| | 029K417-9 | 4800 |
| | 029K417-10 | 5500 |
| | 029K417-11 | 4900 |
| Novel sub-lines of | 029K418-1 | 7900 |
| 029K418 | 029K418-2 | 7500 |
| Novel sub-lines of | 037K421-1 | 9500 |
| 037K421 | 037K421-2 | 6700 |
| | 037K421-3 | 7900 |
| | 037K421-4 | 8500 |
| | 037K421-5 | 9300 |
| | 037K421-6 | 7000 |
| | 037K421-7 | 6700 |
| | 037K421-8 | 7100 |
| | 037K421-9 | 8500 |
| | 037K421-10 | 11200 |
| | 037K421-11 | 7300 |
| | 037K421-12 | 7700 |
| | 037K421-13 | 7900 |
| | 037K421-14 | 9000 |
| Novel sub-lines of | 031K420-1 | 6700 |
| 031K420 | 031K420-2 | 7900 |
| | 031K420-3 | 7400 |
| | 031K420-4 | 9500 |
| | 031K420-5 | 6900 |
| | 031K420-6 | 7100 |

### Genetic Determinant of Ultra-Low Trypsin Inhibitor Phenotype

In order to determine the genetic mechanism of the ultra-low trypsin inhibitor phenotype, data from several crosses in 2015 was analyzed. Table 2 shows data from three soybean populations, SN4591, SV1429, and SV1578. The data were organized into 4 classes according to TIU content. Class one included soybeans having a TIU content of 35,000 or greater; class two included soybeans having a TIU content of 25,000 to 35,000; class three included soybeans having a TIU content of 15,000 to 25,000; and class four included soybeans having a TIU content of less than 15,000. Column one shows the population, column two shows the suspected genetic model or mechanism, column three shows the sample size, columns 4-7 show the actual data , columns 8-11 show the expected data frequency for the predicted genetic model, and column 12 shows the X² statistic P value for a Chi-Square analysis. Models are generally accepted if the P value is above 0.05 or 0.10. As shown in Table 2, all three populations are fixed for the Kunitz locus in the K (or low trypsin inhibitor) state. Therefore, based on the data, it is hypothesized that are a total of 4 total genes, including the Kunitz gene, that are responsible for the ultra-low trypsin inhibitor phenotype. This hypothesis is based on the assumption that one of the additive loci that is segregating in all three crosses is in common among all populations, which leaves one additional additive locus that is in common and segregating among the SN4591 and SV1578 populations, and one dominant locus segregating in SV1429. In the model, it was assumed that all hypothesized alleles were all of equal effect. A total of 7 genetic models were tested, and Table 2 shows the best fitting model of inheritance for the ultra-low trypsin inhibitor phenotype.

TABLE 2

| | | | Actual data freq. | | | | Expected data freq. | | | | |
|---|---|---|---|---|---|---|---|---|---|---|---|
| | | | Class | | | | Class | | | | X² P |
| Population | Model | n | 1 | 2 | 3 | 4 | 1 | 2 | 3 | 4 | value |
| SV1429 | 1 dominant | 45 | 5 | 28 | 10 | 2 | 5 | 30 | 10 | 0 | 0.936 |
| (T3311-1/2* | and 1 additive | | | | | | | | | | |
| 29T516P) | locus | | | | | | | | | | |
| SN4591 | 2 additive | 84 | 8 | 65 | 11 | 0 | 9.33 | 65.33 | 9.33 | 0 | 0.783 |
| (T3311-1/2* | loci | | | | | | | | | | |
| 4C88746) | | | | | | | | | | | |
| SV1578 | 2 additive | 51 | 9 | 35 | 7 | 0 | 5.67 | 39.67 | 5.67 | 0 | 0.244 |
| (29T516P* | loci | | | | | | | | | | |
| 4C88816) | | | | | | | | | | | |

Table 3 below shows 15 soybean samples that were analyzed in spring 2015 for 2S-Protein content, including Kuntiz trypsin inhibitor (KTI), Bowman-Birk inhibitor (BBI), and all other proteins (which are mainly comprised of smaller proteins than the BBI). TSP stands for total soluble protein and is measured as a percentage of the total soluble 2S-Protein. Note sample numbers 4, 6, 14, and 15, expressing ultra-low levels of trypsin inhibitor protein in various embodiments of the invention when compared, for example, to commercial samples 1-3. Total soluble protein was measured on defatted soybeans using the standard technique of polyacrylamide gel electrophoresis, which is well-known in the art.

TABLE 3

| | | 2S-Proteins | | | |
|---|---|---|---|---|---|
| Sample | | KTI (% TSP) | BBI (% TSP) | others (% TSP) | total (% TSP) |
| 1 | 93B82 | 7.38 ± 0.23 | 2.38 ± 0.55 | 2.33 ± 0.09 | 12.08 ± 0.23 |
| 2 | Kunitz | 1.50 ± 0.04 | 1.55 ± 0.05 | 2.33 ± 0.84 | 5.38 ± 0.93 |
| 3 | 435.TCS | 5.37 ± 0.17 | 2.04 ± 0.14 | 2.34 ± 0.02 | 9.75 ± 0.32 |
| 4 | 26T512P4C92361IAHC | 0.55 ± 0.23 | 1.22 ± 0.09 | 3.28 ± 0.37 | 5.05 ± 0.70 |
| 5 | XC2782 | 5.28 ± 0.19 | 1.94 ± 0.01 | 1.69 ± 0.24 | 8.91 ± 0.06 |
| 6 | T3311-1/2IACB | 1.09 ± 0.08 | 0.70 ± 0.06 | 3.35 ± 0.18 | 5.14 ± 0.19 |
| 7 | Y38y207P | 4.68 ± 0.14 | 2.03 ± 0.42 | 3.15 ± 0.31 | 9.86 ± 0.88 |
| 8 | L3011IACB | 4.58 ± 0.14 | 2.28 ± 0.22 | 1.95 ± 0.03 | 8.81 ± 0.04 |
| 9 | XY2162 | 5.39 ± 0.34 | 1.46 ± 0.64 | 1.54 ± 0.64 | 8.40 ± 2.62 |
| 10 | 435P202 | 5.69 ± 0.15 | 2.04 ± 0.28 | 2.22 ± 0.27 | 9.95 ± 0.15 |
| 11 | XP3782S | 5.56 ± 1.23 | 2.21 ± 0.77 | 2.57 ± 0.24 | 10.34 ± 1.76 |
| 12 | 389F.YC | 5.03 ± 0.75 | 1.36 ± 0.24 | 2.37 ± 1.59 | 9.29 ± 1.57 |
| 13 | 439P208 | 4.69 ± 0.05 | 2.69 ± 0.09 | 2.94 ± 0.17 | 10.32 ± 2.21 |
| 14 | 29T514PIAEH | 1.86 ± 0.11 | 0.78 ± 0.04 | 2.01 ± 0.37 | 4.65 ± 0.44 |
| 15 | 23T507321T705IACH | 2.03 ± 0.09 | 1.04 ± 0.07 | 2.69 ± 0.93 | 5.75 ± 1.10 |

Table 4 below shows proprietary soybean variety designations illustrative of embodiments of the invention and their corresponding trypsin inhibitor unit content, TIU. TIU values were calculated by Eurofins according to methods well-known in the art.

TABLE 4

| Variety | TIU |
|---|---|
| 50103 | 9600 |
| 50104 | 13250 |
| 50105 | 8300 |
| 50107 | 11850 |
| 50109 | 13200 |
| 50110 | 8500 |
| 50111 | 10200 |
| 50114 | 8300 |
| 50115 | 13200 |
| 50116 | 8500 |
| 50117 | 12700 |
| 50119 | 14100 |
| 50121 | 15300 |
| 50125 | 13850 |
| 50126 | 13000 |
| 50128 | 10200 |
| 50131 | 10667 |
| 50132 | 12050 |
| 50137 | 14200 |
| 50147 | 13000 |
| 50189 | 8500 |
| 50190 | 15122 |
| 50237 | 15122 |
| 50243 | 8500 |
| 50244 | 15122 |

### Molecular Techniques and Terminology

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook, et al., Molecular Cloning, Second Edition, Cold Spring Harbor Laboratory, Plainview, N.Y. (1989); Maniatis, et al., Molecular Cloning, Cold Spring Harbor Laboratory, Plainview, N.Y. (1982); Wu (ed.), Meth. Enzymol., 218, Part I (1993); Wu (ed.), Meth. Enzymol., 68 (1979); Wu, et al. (eds.), Meth. Enzymol., 100 and 101 (1983); Grossman and Moldave (eds.), Meth. Enzymol., 65; Miller (ed.), Experiments in Molecular Genetics, Cold Spring Harbor Laboratory, Cold Spring Harbor, N.Y. (1972); Old and Primrose, Principles of Gene Manipulation, University of California Press, Berkley (1981); Schleif and Wensink, Practical Methods in Molecular Biology (1982); Glover (ed.), DNA Cloning, Vols. I and II, IRL Press, Oxford, UK (1985); Hames and Higgins (eds.), Nucleic Acid Hybridization, IRL Press, Oxford, UK (1985); Setlow and Hollaender, Genetic Engineering: Principles and Methods, Vols. 1-4, Plenum Press, New York (1979); and Ausubel, et al., Current Protocols in Molecular Biology, Greene/Wiley, N.Y. (1992). Abbreviations and nomenclature, where employed, are deemed standard in the field and commonly used in professional journals such as those cited herein. Intermediate cloning of the PCR products can be done using the PCRTerminator end repair kit and CLoneSmart kit vector pSMART (Lucigen, Middleton, Wis.). Various PCR based cloning methods are known to those skilled in the art.

It is well-known in the art that the nucleic acid sequences of the present invention can be truncated and/or mutated such that certain of the resulting fragments and/or mutants of the original full-length sequence can retain the desired characteristics of the full-length sequence. A wide variety of restriction enzymes which are suitable for generating fragments from larger nucleic acid molecules are well-known. In addition, it is well-known that BAL 31 exonuclease can be conveniently used for time-controlled limited digestion of DNA. See, for example, Maniatis, Molecular Cloning: A Laboratory Manual, Cold Spring Harbor Laboratory, New York, pp. 135-139 (1982), incorporated herein by reference. See also, Wei, et al., J. Biol. Chem., 258:13006-13512 (1983). By use of BAL 31 exonuclease (commonly referred to as Aerase-a-base procedures), the ordinarily skilled artisan can remove nucleotides from either or both ends of the subject nucleic acids to generate a wide spectrum of fragments which are functionally equivalent to the subject nucleotide sequences. One of ordinary skill in the art can, in this manner, generate hundreds of fragments of controlled, varying lengths from locations all along a starting nucleotide sequence. The ordinarily skilled artisan can routinely test or screen the generated fragments for their characteristics and determine the utility of the fragments as taught herein. It is also well-known that the mutant sequences of the full length sequence, or fragments thereof, can be easily produced with site directed mutagenesis. See, for example, Larionov, O. A. and Nikiforov, V. G., Genetika, 18(3):349-59 (1982); Shortie, D., DiMaio, D., and Nathans, D., Annu. Rev. Genet., 15:265-94 (1981); both incorporated herein by reference. The skilled artisan can routinely produce deletion-, insertion-, or substitution-type mutations and identify those resulting mutants which contain the desired characteristics of the full length wild-type sequence, or fragments thereof, i.e., those which retain promoter activity. It is well-known in the art that there are a variety of other PCR-mediated methods, such as overlapping PCR that may be used.

A nucleic acid sequence or polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

A nucleotide sequence is operably linked when it is placed into a functional relationship with another nucleotide sequence. For instance, a promoter is operably linked to a coding sequence if the promoter effects the transcription or expression of the coding sequence. Operably linked also means that the sequences being linked are contiguous and, where necessary to join two protein coding regions, contiguous and in reading frame; and for example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation. However, it is well-known that certain genetic elements, such as enhancers, may be operably linked even at a distance, i.e., even if not contiguous.

For efficient expression, the coding sequences are preferably also operatively linked to a 3′ untranslated sequence. The 3′ untranslated sequence will include a transcription termination sequence and a polyadenylation sequence. The 3′ untranslated region can be obtained from the flanking regions of genes from Agrobacterium, plant viruses, plants or other eukaryotes. Suitable 3′ untranslated sequences for use in plants include, but are not limited to, those from the cauliflower mosaic virus 35S gene, the phaseolin seed storage protein gene, the pea ribulose biphosphate carboxylase small subunit E9 gene, the soybean 7S storage protein genes, the octopine synthase gene, and the nopaline synthase gene.

Suitable constitutive promoters for use in plants include promoters from plant viruses, such as the peanut chlorotic streak caulimovirus (PC1SV) promoter (U.S. Pat. No. 5,850,019), the 35S promoter from cauliflower mosaic virus (CaMV) (Odell, et al., Nature, 313:810-812 (1985)), promoters of Chlorella virus methyltransferase genes (U.S. Pat. No. 5,563,328), and the full-length transcript promoter from figwort mosaic virus (FMV) (U.S. Pat. No. 5,378,619); the promoters from such genes as rice actin (McElroy, et al., Plant Cell, 2:163-171 (1990)), ubiquitin (Christensen, et al., Plant Mol. Biol., 12:619-632 (1989) and Christensen, et al., Plant Mol. Biol., 18:675-689 (1992)), pEMU (Last, et al., Theor. Appl. Genet., 81:581-588 (1991)), MAS (Velten, et al., EMBO J., 3:2723-2730 (1984)), maize H3 histone (Lepetit, et al., Mol. Gen. Genet., 231:276-285 (1992) and Atanassova, et al., Plant Journal, 2(3):291-300 (1992)), Brassica napes ALS3 (WO 97/41228); and promoters of various Agrobacterium genes (see, U.S. Pat. Nos. 4,771,002, 5,102,796, 5,182,200, and 5,428,147). Finally, promoters composed of portions of other promoters and partially or totally synthetic promoters can be used. See, for example, Ni, et al., Plant J., 7:661-676 (1995) and WO 95/14098 describing such promoters for use in plants.

The promoter may include, or be modified to include, one or more enhancer elements. Preferably, the promoter will include a plurality of enhancer elements. Promoters containing enhancer elements provide for higher levels of transcription as compared to promoters that do not include them. Suitable enhancer elements for use in plants include the PCNV enhancer element (U.S. Pat. No. 5,850,019), the CaMV 35S enhancer element (U.S. Pat. Nos. 5,106,739 and 5,164,316), and the FMV7 enhancer element (Maiti, et al., Transgenic Res., 6:143-156 (1997)). See also, WO 96/23898 and Enhancers and Eukaryotic Expression, Cold Spring Harbor Press, Cold Spring Harbor, N.Y. (1983).

The regeneration, development, and cultivation of plants from single plant protoplast transformants or from various transformed explants is also well-known in the art. Weissbach and Weissbach, (eds.), In: Methods for Plant Molecular Biology, Academic Press, Inc., San Diego, Calif. (1988).

There are a variety of methods for the regeneration of plants from plant tissue. The particular method of regeneration will depend on the starting plant tissue and the particular plant species to be regenerated.

Methods for transformation are well-known and have been published for soybean (U.S. Pat. Nos. 6,576,820, 5,569,834, 5,416,011; McCabe, et. al., BiolTechnology, 6:923 (1988); Christou, et al., Plant Physiol., 87:671-674 (1988)); cotton (U.S. Pat. Nos. 5,004,863, 5,159,135, 5,518,908); Brassica (U.S. Pat. No. 5,463,174); peanut (Cheng, et al., Plant Cell Rep., 15:653-657 (1996); McKently, et al., Plant Cell Rep., 14:699-703 (1995)); papaya and pea (Grant, et al., Plant Cell Rep., 15:254-258 (1995)).

A DNA construct can be used to transform any type of plant or plant cell. A genetic marker can be used for selecting transformed plant cells (“a selection marker”). Selection markers typically allow transformed cells to be recovered by negative selection (ie, inhibiting growth of cells that do not contain the selection marker) or by screening for a product encoded by the selection marker. The most commonly used selectable marker gene for plant transformation is the neomycin phosphotransferase II (nptII) gene, isolated from Tn5, which, when placed under the control of plant expression control signals, confers resistance to kanamycin. Fraley, et al., Proc. Natl. Acad. Sci. USA, 80:4803 (1983). Additional selectable marker genes of bacterial origin that confer resistance to antibiotics include gentamycin acetyl transferase, streptomycin phosphotransferase, aminoglycoside-3′-adenyl transferase, and the bleomycin resistance determinant. Hayford, et al., Plant Physiol., 86:1216 (1988); Jones, et al., Mol. Gen. Genet., 210:86 (1987); Svab, et al., Plant Mol. Biol., 14:197 (1990); Hille, et al., Plant Mol. Biol., 7:171 (1986). Other selectable marker genes confer resistance to herbicides, such as glyphosate, glufosinate, or bromoxynil. Comai, et al., Nature, 317:741-744 (1985); Stalker, et al., Science, 242:419-423 (1988); Hinchee, et al., Bio/Technology, 6:915-922 (1988); Stalker, et al., J. Biol. Chem., 263:6310-6314 (1988); and Gordon-Kamm, et al., Plant Cell, 2:603-618 (1990).

Additional selectable markers useful for plant transformation include, without limitation, mouse dihydrofolate reductase, plant 5-enolpyruvylshikimate-3-phosphate synthase, and plant acetolactate synthase. Eichholtz, et al., Somatic Cell Mol. Genet., 13:67 (1987); Shah, et al., Science, 233:478 (1986); Charest, et al., Plant Cell Rep., 8:643 (1990); EP 154,204.

Commonly used genes for screening presumptively transformed cells include, but are not limited to, beta.-glucuronidase (GUS), beta.-galactosidase, luciferase, and chloramphenicol acetyltransferase (Jefferson, R. A., Plant Mol. Biol. Rep., 5:387 (1987); Teen, et al., EMBO J., 8:343 (1989); Koncz, et al., Proc. Natl. Acad. Sci. USA, 84:131 (1987); De Block, et al., EMBO J., 3:1681 (1984)), green fluorescent protein (GFP) and its variants (Chalfie, et al., Science, 263:802 (1994); Haseloff, et al., TIG, 11:328-329 (1995), and WO 97/41228). Another approach to the identification of relatively rare transformation events has been use of a gene that encodes a dominant constitutive regulator of the Zea mays anthocyanin pigmentation pathway (Ludwig, et al., Science, 247:449 (1990)).

Assays for gene expression based on the transient expression of cloned nucleic acid constructs have been developed by introducing the nucleic acid molecules into plant cells by polyethylene glycol treatment, electroporation, or particle bombardment (Marcotte, et al., Nature, 335:454-457 (1988); Marcotte, et al., Plant Cell, 1:523-532 (1989); McCarty, et al., Cell, 66:895-905 (1991); Hattori, et al., Genes Dev., 6:609-618 (1992); Goff, et al., EMBO J., 9:2517-2522 (1990)).

Transient expression systems may be used to functionally dissect isolated nucleic acid fragment constructs (see generally, Maliga, et al., Methods in Plant Molecular Biology, Cold Spring Harbor Press (1995)). It is understood that any of the nucleic acid molecules of the present invention can be introduced into a plant cell in a permanent or transient manner in combination with other genetic elements such as vectors, promoters, enhancers, etc.

In addition to the above discussed procedures the standard resource materials which describe specific conditions and procedures for the construction, manipulation and isolation of macromolecules (e.g., DNA molecules, plasmids, etc.), generation of recombinant organisms and screening and isolating of clones (see, for example, Sambrook, et al., Molecular Cloning: A Laboratory Manual, Cold Spring Harbor Press (1989); Maliga, et al., Methods in Plant Molecular Biology, Cold Spring Harbor Press (1995); Birren, et al., Genome Analysis: Detecting Genes, 1, Cold Spring Harbor, N.Y. (1998); Birren, et al., Genome Analysis: Analyzing DNA, 2, Cold Spring Harbor, N.Y. (1998); Clark, Springer (eds.), Plant Molecular Biology: A Laboratory Manual, New York (1997)) are well-known.

It is recognized by those skilled in the art that the DNA sequences may vary due to the degeneracy of the genetic code and codon usage.

### Conventional Breeding Techniques, Terminology, and Use of Molecular Markers

The term “backcrossing” as used herein refers to the repeated crossing of progeny back to the recurrent parent, i.e., backcrossing 1, 2, 3, 4, 5, 6, 7, 8, or more times to the recurrent parent. The parental soybean plant that contributes the gene for the desired characteristic is termed the nonrecurrent or donor parent. This terminology refers to the fact that the nonrecurrent parent is used one time in the backcross protocol and therefore does not recur. The parental soybean plant to which the gene or genes from the nonrecurrent parent are transferred is known as the recurrent parent as it is used for several rounds in the backcrossing protocol (Poehlman & Sleper (1994); Fehr, Principles of Cultivar Development, pp. 261-286 (1987)). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a soybean plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original variety. To accomplish this, a single gene of the recurrent variety is modified or substituted with the desired gene from the nonrecurrent parent, while retaining essentially all of the rest of the desired genetic, and therefore the desired physiological and morphological, constitution of the original variety. The choice of the particular nonrecurrent parent will depend on the purpose of the backcross; one of the major purposes is to add some agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new variety but that can be improved by backcrossing techniques. Single gene traits may or may not be transgenic. Examples of these traits include, but are not limited to, male sterility, waxy starch, herbicide resistance, resistance for bacterial, fungal, or viral disease, insect resistance, male fertility, enhanced nutritional quality, industrial usage, yield stability, and yield enhancement. These genes are generally inherited through the nucleus. Several of these single gene traits are described in U.S. Pat. Nos. 5,959,185, 5,973,234, and 5,977,445, the disclosures of which are specifically hereby incorporated by reference.

A backcross conversion of soybean plants of the present invention occurs when DNA sequences are introduced through backcrossing (Hallauer, et al., “Corn Breeding,” Corn and Corn Improvements, No. 18, pp. 463-481 (1988)). Both naturally occurring and transgenic DNA sequences may be introduced through backcrossing techniques. A backcross conversion may produce a plant with a trait or locus conversion in at least two or more backcrosses, including at least two crosses, at least three crosses, at least four crosses, at least five crosses, and the like. Molecular marker assisted breeding or selection may be utilized to reduce the number of backcrosses necessary to achieve the backcross conversion. For example, see, Openshaw, S. J., et al., Marker-assisted Selection in Backcross Breeding, In: Proceedings Symposium of the Analysis of Molecular Data, Crop Science Society of America, Corvallis, Oreg. (August 1994), where it is demonstrated that a backcross conversion can be made in as few as two backcrosses.

The complexity of the backcross conversion method depends on the type of trait being transferred (single genes or closely linked genes as vs. unlinked genes), the level of expression of the trait, the type of inheritance (cytoplasmic or nuclear) and the types of parents included in the cross. It is understood by those of ordinary skill in the art that for single gene traits that are relatively easy to classify, the backcross method is effective and relatively easy to manage. (See, Hallauer, et al., Corn and Corn Improvement, Sprague and Dudley, Third Ed. (1998)). In addition, an introgression site itself, such as an FRT site, Lox site or other site specific integration site, may be inserted by backcrossing and utilized for direct insertion of one or more genes of interest into a specific plant variety. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of site specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes transferred via backcrossing typically function as a dominant single gene trait and are relatively easy to classify. Selection of progeny for a trait that is transferred via a recessive allele requires growing and selling the first backcross generation to determine which plants carry the recessive alleles. Recessive traits may require additional progeny testing in successive backcross generations to determine the presence of the locus of interest. The last backcross generation is usually selfed to give pure breeding progeny for the gene(s) being transferred, although a backcross conversion with a stably introgressed trait may also be maintained by further backcrossing to the recurrent parent with selection for the converted trait.

Along with selection for the trait of interest, progeny are selected for the desired phenotype. The backcross is a form of inbreeding, and the features of the recurrent parent are automatically recovered after successive backcrosses. Poehlman, Breeding Field Crops, p. 204 (1987). Poehlman suggests from one to four or more backcrosses, but as noted above, the number of backcrosses necessary can be reduced with the use of molecular markers. Other factors, such as a genetically similar donor parent, may also reduce the number of backcrosses necessary. As noted by Poehlman, backcrossing is easiest for simply inherited, dominant and easily recognized traits.

The new soybean plants of the present invention also provide a source of breeding material that may be used to develop other new soybean varieties. Plant breeding techniques known in the art and used in a soybean plant breeding program include, but are not limited to, recurrent selection, mass selection, bulk selection, mass selection, backcrossing, pedigree breeding, open pollination breeding, restriction fragment length polymorphism enhanced selection, genetic marker enhanced selection, making double haploids, and transformation. Often combinations of these techniques are used. The development of soybean varieties in a plant breeding program requires, in general, the development and evaluation of homozygous varieties. There are many analytical methods available to evaluate a new variety. The oldest and most traditional method of analysis is the observation of phenotypic traits but genotypic analysis may also be used.

Additional embodiments are directed to methods for producing a soybean plant by crossing a first parent soybean plant with a second parent soybean plant wherein either the first or second parent soybean plant of the present invention. Further, another embodiment of the present invention is also directed to methods for producing a soybean plant by crossing a soybean plant of the present invention with a second soybean plant and growing the progeny seed, and repeating the crossing and growing steps with the soybean plant of the present invention from 1, 2, 3, 4, 5, 6 to 7 times. Thus, any such methods using a soybean plant of the present invention are part of this invention: selfing, backcrosses, hybrid production, crosses to populations, and the like. All plants produced using a soybean plant of the present invention as a parent are within the scope of this invention, including plants derived from soybean plants of the present invention. These methods are well-known in the art and some of the more commonly used breeding methods are described below. Descriptions of breeding methods can be found in one of several reference books (e.g., Allard, Principles of Plant Breeding (1960); Simmonds, Principles of Crop Improvement (1979); Sneep, et al., (1979); Fehr, “Breeding Methods for Cultivar Development,” Chapter 7, Soybean Improvement, Production and Uses, 2nd ed., Wilcox editor (1987).

The following describes breeding methods that may be used with the soybean plants of the present invention in the development of further soybean plants. One such embodiment is comprised of: obtaining the soybean plant, or a part thereof, of the soybean plant of the present invention, utilizing said plant or plant part as a source of breeding material and selecting a soybean progeny plants with molecular markers in common with soybean plants of the present invention and/or with morphological and/or physiological. Breeding steps that may be used in the soybean plant breeding program include pedigree breeding, backcrossing, mutation breeding, and recurrent selection. In conjunction with these steps, techniques such as RFLP-enhanced selection, genetic marker enhanced selection (for example, SSR markers) and the making of double haploids may be utilized.

One of ordinary skill in the art of plant breeding would know how to evaluate the traits of soybean plants of the present invention to determine if there is no significant difference between the traits expressed by the soybean plants of the present invention and other soybean plants.

Pedigree breeding starts with the crossing of two genotypes if the two original parents do not provide all the desired characteristics, other sources can be included in the breeding population. In the pedigree method, superior plants are selfed and selected in successive filial generations. In the succeeding filial generations the heterozygous condition gives way to homogeneous varieties as a result of self-pollination and selection. Typically in the pedigree method of breeding, five or more successive filial generations of selfing and selection is practiced: F₁ to F₂; F₂ to F₃; F₃ to F₄; F₄ to F₅; etc. After a sufficient amount of inbreeding, successive filial generations will serve to increase seed of the developed variety.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent but at the same time retain many components of the nonrecurrent parent by stopping the backcrossing at an early stage and proceeding with selfing and selection. For example, a soybean variety may be crossed with another variety to produce a first generation progeny plant. The first generation progeny plant may then be backcrossed to one of its parent varieties to create a BC1 or BC2. Progeny are selfed and selected so that the newly developed variety has many of the attributes of the recurrent parent and yet several of the desired attributes of the nonrecurrent parent. This approach leverages the value and strengths of the recurrent parent for use in new soybean varieties.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny and selfed progeny. The selected progeny are cross pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the intercrossing of several selected varieties.

Mass selection is a useful technique when used in conjunction with molecular marker enhanced selection. In mass selection seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Also, instead of self-pollination, directed pollination could be used as part of the breeding program.

Traditional breeding techniques can be enhanced through the use of molecular markers, which includes markers identified through the use of techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods utilizing the soybean plants of the present invention.

Isozyme Electrophoresis and RFLPs have been widely used to determine genetic composition. Shoemaker and Olsen, Molecular Linkage Map of Soybean (Glycine max L. Merr.), pp. 6.131-6.138 (1993). In S. J. O'Brien, (ed.), Genetic Maps: Locus Maps of Complex Genomes, Cold Spring Harbor Laboratory Press, Cold Spring Harbor, N.Y., developed a molecular genetic linkage map that consisted of twenty-five linkage groups with about 365 RFLP, 11 RAPD (random amplified polymorphic DNA), three classical markers, and four isozyme loci. See also, Shoemaker, R. C., RFLP Map of Soybean, pp. 299-309 (1994); In Phillips, R. L. and Vasil, I. K. (ed.), DNA-based markers in plants, Kluwer Academic Press Dordrecht, the Netherlands.

SSR technology is currently the most efficient and practical marker technology; more marker loci can be routinely used and more alleles per marker locus can be found using SSRs in comparison to RFLPs. For example, Diwan and Cregan described a highly polymorphic microsatellite loci in soybean with as many as 26 alleles. (Diwan, N. and Cregan, P. B., Automated sizing of fluorescent-labelled simple sequence repeat (SSR) markers to assay genetic variation in Soybean, Theor. Appl. Genet., 95:220-225 (1997). Single Nucleotide Polymorphisms may also be used to identify the unique genetic composition of the invention and progeny varieties retaining that unique genetic composition. Various molecular marker techniques may be used in combination to enhance overall resolution.

Soybean DNA molecular marker linkage maps have been rapidly constructed and widely implemented in genetic studies. One such study is described in Cregan et al, “An Integrated Genetic Linkage Map of the Soybean Genome,” Crop Science, 39:1464-1490 (1999). Sequences and PCR conditions of SSR Loci in Soybean as well as the most current genetic map may be found in Soybase on the World Wide Web.

One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses.

### Commercial Use of Soybean and Soybean Products

Industrial uses of soybean oil which is subjected to further processing include ingredients for paints, plastics, fibers, detergents, cosmetics, lubricants, and biodiesel fuel. Soybean oil may be split, inter-esterified, sulfurized, epoxidized, polymerized, ethoxylated, or cleaved. Designing and producing soybean oil derivatives with improved functionality and improved oliochemistry is a rapidly growing field. The typical mixture of triglycerides is usually split and separated into pure fatty acids, which are then combined with petroleum-derived alcohols or acids, nitrogen, sulfonates, chlorine, or with fatty alcohols derived from fats and oils.

Soybean is also used as a food source for both animals, aquaculture, and humans. Soybean is widely used as a source of protein for animal feeds for poultry, swine and cattle.

For human consumption soybean meal is made into soybean flour which is processed to protein concentrates used for meat extenders, aquaculture, or specialty pet foods. Production of edible protein ingredients from soybean offers a healthier, less expensive replacement for animal protein in meats as well as in dairy-type products. Alternatively, insome embodiments, the food product may comprise beverages such as soymilk and other nutritional beverages, infused foods, sauces, condiments, salad dressings, fruit juices, syrups, desserts, icings and fillings, soft frozen products, confections, or intermediate foods. Foods produced from the plants of the invention may comprise reduced trypsin inhibitor content and thus be of greater nutritional value than foods made with typical soybean varieties.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

### DEPOSIT INFORMATION

A deposit of the Schillinger Genetics, Inc. soybean seed exhibiting the ultra-low trypsin inhibitor unit phenotype and genotype described within this application has been made with American Type Culture Collection (ATCC), 10801 University Boulevard, Manassas, Va. 20110. The date of deposit was Feb. 24, 2010. The deposit of seed were taken from the same deposit maintained by Schillinger Genetics, Inc. since prior to the filing date of this application. All restrictions will be removed upon granting of a patent, and the deposit is intended to meet all of the requirements of 37 C. F. R. §§1.801-1.809. The ATCC accession number is PTA-10684. The deposit will be maintained in the depository for a period of thirty years, or five years after the last request, or for the enforceable life of the patent, whichever is longer, and will be replaced as necessary during the period.

1. An untreated and unheated soybean seed that expresses a diminished amount of an endogenous 2S-Protein, wherein said endogenous 2S-Protein is a protease trypsin inhibitor, and wherein the amount of trypsin inhibitor units within said seed is between 4,800 trypsin inhibitor units and 15,122 trypsin inhibitor units.

2. A plant, or part thereof, produced by growing the seed of claim 1.

3. The soybean seed of claim 1, wherein the total soluble 2S-Protein is approximately 6.85% or less.

4. The soybean seed of claim 3, wherein the amount of Kunitz trypsin inhibitor protein is approximately 3.01% or less of the total soluble 2S-Protein.

5. The soybean seed of claim 3, wherein the amount of Bowman-Birk inhibitor protein is approximately 1.31% or less of the total soluble 2S-Protein.

6. A method of producing a commodity plant product, comprising obtaining the plant of claim 2, or a part thereof, wherein the commodity plant product is protein concentrate, protein isolate, soybean hulls, meal, flour or oil and producing said commodity plant product therefrom.

7. A method for introducing the combination of the Kunitz allele and three alleles associated with conferring a ultra-low trypsin inhibitor phenotype to a soybean plant lacking said alleles comprising:
(a) obtaining a first soybean plant wherein said soybean plant contains a genome comprising the Kunitz allele and three alleles associated with conferring a ultra-low trypsin inhibitor phenotype, wherein a representative sample of said alleles is present in ATCC accession number is PTA-10684;
(b) crossing said first soybean plant with a second soybean plant, wherein said second soybean plant lacks said alleles;
(c) selecting for progeny plants that have low trypsin inhibitor unit content; and
(d) backcrossing said progeny plants to said first parent plant until the progeny plants can be identified as exhibiting the Kunitz allele and three alleles associated with the ultra-low trypsin inhibitor phenotype.

8. A seed produced by growing the plant of the method of claim 7.

9. A method for producing a soybean seed, comprising crossing two soybean plants and harvesting the resultant soybean seed, wherein at least one soybean plant is the plant of claim 2.

10. A method of producing a commodity plant product, comprising obtaining the plant of claim 7, or a part thereof, wherein the commodity plant product is protein concentrate, protein isolate, soybean hulls, meal, flour or oil and producing said commodity plant product therefrom.

11. An untreated and unheated soybean seed that expresses a diminished amount of a protease trypsin inhibitor, wherein the amount of trypsin inhibitor units within said seed is between 4,800 trypsin inhibitor units and 15,122 trypsin inhibitor units, and wherein said diminished amount of a protease trypsin inhibitor is due to the presence of the Kunitz allele and at least three additional alleles, wherein a representative sample of said alleles is present in ATCC accession number is PTA-10684.

12. The three additional alleles of claim 11, wherein the alleles are comprised of a dominant allele and two additive alleles.
